(19) United States
(12) Patent Application Publication
(10) Pub. No.: US 2026/0160745 A1
(43) Pub. Date: Jun. 11, 2026

(54) SENSOR AND ARRANGEMENT

(71) Applicant: Volkswagen Aktiengesellschaft, Wolfsburg (DE)

(72) Inventor: Fabian BREMER, Wolfsburg (DE)

(73) Assignee: Volkswagen Aktiengesellschaft, Wolfsburg (DE)

(21) Appl. No.: 19/410,453

(22) Filed: Dec. 5, 2025

(30) Foreign Application Priority Data
Dec. 6, 2024 (DE) 10 2024 136 563.3

(51) Int. Cl. G01N 33/00

(52) U.S. Cl. CPC G01N 33/0073; G01N 33/004; G01N 33/005

(57) ABSTRACT

A sensor for monitoring a measured variable. The sensor has a supply voltage input, a sensor output, and a system state input. The sensor is designed, for a switched-on system, to generate a first signal, having a first constant level, at the sensor output and when a measured variable greater than a threshold value is detected, to generate at least one second signal having at least one second level. For a switched-off system the sensor is designed to switch into a sleep mode and to generate no signal at the sensor output, and to periodically wake up and detect the measured variable. In the awakened state at least one first signal having the first level is present at the sensor output. The invention further relates to an arrangement including a sensor and a control unit.

This nonprovisional application claims priority under 35 U.S.C. § 119(a) to German Patent Application No. 10 2024 136 563.3, which was filed in Germany on Dec. 6, 2024, and which is herein incorporated by reference.

### BACKGROUND OF THE INVENTION

### Field of the Invention

The invention relates to a sensor and to an arrangement including such a sensor.

### Description of the Background Art

Sensors are known for a wide variety of applications. Sensors often need a supply voltage, so that they have a corresponding quiescent current requirement. It is therefore known to switch these sensors off when the system in which they are situated is switched off. This problem is exacerbated with sensors that must continuously generate an output signal in order to signal their functional capability. On the other hand, systems exist which have components that could also go into a critical state, even in the switched-off state of the system.

### SUMMARY OF THE INVENTION

It is therefore an object of the present invention to provide a sensor as well as an arrangement including such a sensor, which at least reduce the problem described above.

The sensor for monitoring a measured variable, according to an example, has a supply voltage input, a sensor output, and a system state input. The sensor is designed, for a switched-on system, to generate a first signal, having a first constant level, at the sensor output. This first signal is generated even if no measured variable is detected, and signals the functional capability of sensor, which is then monitored by a control unit, for example. In addition, the sensor, when a measured variable greater than a threshold value is detected, is designed to generate at least one second signal having at least one second level. The second level is higher than the first level. The levels may be analog values (voltage values, for example) or digital values (PWM signals or binary values, for example). Furthermore, for a switched-off system the sensor is designed to switch into a sleep mode and to generate no signal at the sensor output. Accordingly, in this sleep mode the sensor has no, or only a minimal, idle state requirement. The sensor is further designed to periodically wake up, with the measured variable being detected in the awakened state, and at least one first signal having the first level being present. If no measured variable greater than the threshold value is detected, the sensor goes back into sleep mode. The sensor may thus quasi-continuously monitor a component, with the quiescent current requirement being reduced as a result of the sleep mode. The wake-up time may be less than or equal to the sleep time, provided that it is ensured that measurement of the measured variable can be carried out.

The sensor can be designed as a gas sensor that monitors, for example, a gas concentration of a battery cell or battery unit.

It is further preferred that the sensor can be designed as an analog sensor; i.e., at least the levels at the sensor output are analog signals. The advantage of analog sensors is that triggering thresholds may often be adapted more easily, as discussed in greater detail below.

The frequency at which the sensor is periodically awakened is then a function of how quickly the state of a component to be monitored can be changed. For a gas sensor for monitoring battery cells or battery units, the frequency is preferably 0.01-1 Hz, more preferably 0.1-0.5 Hz.

The arrangement includes a control unit and an above-described sensor, with the sensor output being connected to the control unit. The control unit is designed in such a way that for a switched-on system and a signal below the first level, a defective sensor is deduced. The control unit may then, for example, generate a warning message and/or initiate other countermeasures.

The control unit can be designed in such a way that for a switched-on system and a signal above a third level, the third level being higher than the second level, a defective sensor is deduced, for example because a short circuit to the supply voltage is present. The first level and the third level thus define a permissible value range at the sensor output.

The control unit, for a switched-off system, can be designed to be in a sleep mode and to be awakened by a signal that is higher than the second level.

The control unit can be preferably further designed, in the awakened state, to change or switch the system state input of the sensor to “switched-on system.”

The control unit can be designed in such a way that in the switched-on state and with a signal that is higher than the second level (and lower than the third level), it deduces a state of the component, for example that a battery cell is defective and a thermal runaway of a battery unit is imminent.

The control unit can be designed to adapt the levels as a function of a control signal and/or detected measured values. Thus, for example, the control unit can respond to an identified drift of the measured values or of the signals over the service life of the sensor, possibly avoiding frequent false warnings.

The component can be a battery unit, and/or the control unit is a battery management control unit.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 shows a schematic illustration of an arrangement including a sensor and a control unit,

FIG. 2 shows a schematic illustration of a signal at a sensor output over time for a switched-on system, and

FIG. 3 shows a schematic illustration of signal at a sensor output over time for a switched-off system.

### DETAILED DESCRIPTION

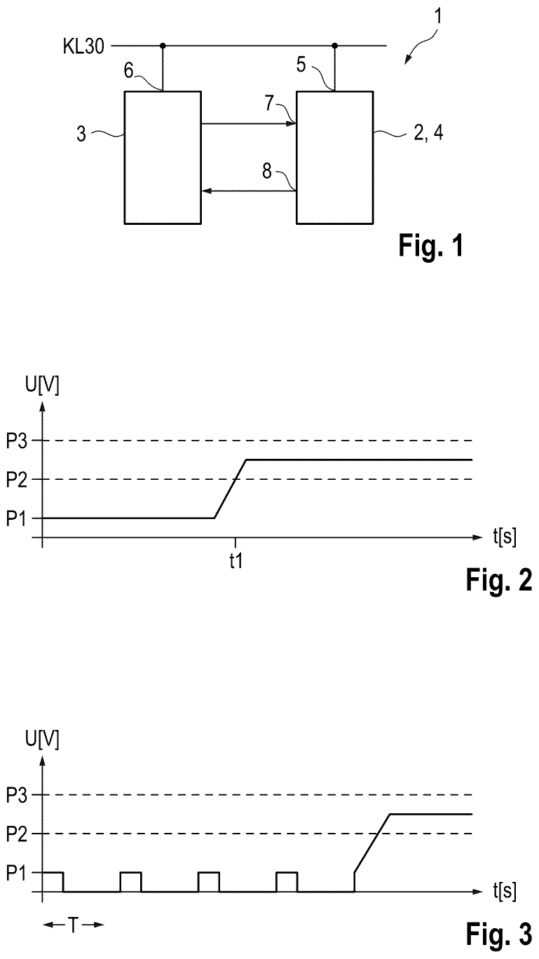

FIG. 1 schematically illustrates an arrangement 1 that includes a sensor 2 and a control unit 3. The sensor 2 is preferably a gas sensor 4. The arrangement 1 is, for example, an integral part of a battery system of a traction network of an electric vehicle, wherein the gas sensor 4 monitors, for example, the CO or H₂ concentration at a battery unit, so that when a battery cell is defective the sensor can initiate countermeasures to avoid a thermal runaway of the other battery cells and generate warning messages. The sensor 2 has a supply voltage input 5 that is connected to a continuous voltage KL30. Likewise, the control unit 3 may have a voltage supply input 6 at which the continuous voltage KL30 is present. The voltage may be provided from the battery unit, to be monitored, by a low-voltage battery and/or via a DC/DC converter.

In addition, the sensor 2 includes a system state input 7 that is connected to the control unit 3, and a sensor output 8 that is likewise connected to the control unit 3. The system state input 7 signals to the sensor 2 whether or not the battery system is switched on, in particular whether or not the control unit 3 is switched on. If the control unit 3 is switched on, a signal is present at the system state input 7. If the sensor 2 is a digital sensor, for example a logical 1 is present. If the sensor 2 is an analog sensor, for example a voltage greater than a threshold value is present. The threshold value is 6 V, for example.

With reference to FIG. 2, the operating principle of the sensor 2 for a switched-on system is first explained. The sensor at its sensor output generates a first voltage signal U having a first constant level P1, even if no measured variable (i.e., no CO or H₂) is detected or the measured variable is below a detection threshold. This signal at the sensor output 8 is read by the control unit 3. As long as this signal having the level P1 is detected, the control unit 3 knows that the sensor 2 is active and is not defective. On the other hand, if the signal drops below the first level P1 or exceeds a third level P3, the control unit 3 deduces that the sensor 2 is defective and carries out countermeasures. For example, if gas then escapes from a defective battery cell of the battery unit, the signal at the sensor output 8 increases, wherein a second level P2 or threshold value is exceeded at time t₁, which the control unit 3 detects as a potential imminent thermal runaway of the battery unit, and control unit takes appropriate measures.

If the system is now switched off, the control unit 3 and the sensor 2 go into a sleep mode, and other components of the system may be completely switched off. For example, a voltage below the threshold value (less than 6 V, for example) is then present at the system state input 7. The sensor 2 is designed to periodically wake itself up and perform a measurement of the measured variable (the CO or H₂ concentration, for example). In the phases in which the sensor 2 is awakened, once again a voltage having a level P1 is present at the sensor output 8. The phase in which the sensor 2 is awakened is shorter than the phase in which the sensor 2 is in sleep mode, and the voltage at the sensor output 7 is zero. This is illustrated in FIG. 3. The period duration T is the reciprocal of the frequency at which the sensor 2 is periodically awakened. If the sensor 2 then detects a measured variable greater than a threshold value, the sensor generates a signal at the sensor output 8 that is higher than the second level P2. This signal, which is higher than the second level P2, is a wake-up signal for the control unit 3. The control unit 3 generates a signal at the system state input 7 that the system is switched on. In addition, the control unit 3 carries out measures, for example generating a warning message or initiating further measurements, to verify the risk of a thermal runaway. As a result of the sleep mode with periodic self-waking, the sensor 2 quasi-continuously monitors the component, and the quiescent current requirement is reduced significantly.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A sensor for monitoring a measured variable, the sensor comprising:
a supply voltage input;
a sensor output; and
a system state input,
wherein the sensor is designed, for a switched-on system, to generate a first signal, having a first constant level, at the sensor output, and when a measured variable greater than a threshold value is detected, to generate at least one second signal having at least one second level, and
wherein for a switched-off system, the sensor is designed to switch into a sleep mode and to generate no signal at the sensor output, and to periodically wake up and detect the measured variable, wherein, in an awakened state, at least one first signal having the first level is present at the sensor output.

2. The sensor according to claim 1, wherein the sensor is a gas sensor.

3. The sensor according to claim 1, wherein the sensor an analog sensor.

4. An arrangement comprising:
a control unit; and
the sensor according to claim 1,
wherein the sensor output of the sensor is connected to the control unit,
wherein the control unit is designed such that for the switched-on system and a signal below the first level, a defective sensor is deduced.

5. The arrangement according to claim 4, wherein the control unit is designed in such a way that for a switched-on system and a signal above a third level, which is higher than the second level, a defective sensor is deduced.

6. The arrangement according to claim 4, wherein the control unit, for a switched-off system, is designed to be in a sleep mode and to be awakened by a signal that is higher than the second level at the sensor output.

7. The arrangement according to claim 6, wherein the control unit is designed, in the awakened state, to switch the system state input to a switched-on system.

8. The arrangement according to claim 4, wherein the control unit is designed in such a way that in the switched-on state and with a signal that is higher than the second level, it deduces a state of a component.

9. The arrangement according to claim 4, wherein the control signal is designed to adapt the levels as a function of a control signal and/or detected measured values.

10. The arrangement according to claim 8, wherein the component is a battery unit, and/or the control unit is a battery management control unit.
